(19) United States
(12) Patent Application Publication
(10) Pub. No.: US 2012/0271431 A1
(43) Pub. Date: Oct. 25, 2012

(54) Bioactively Coated Metal Implants and Methods for the Production Thereof

(75) Inventors: Berthold Nies, Frankisch-Crumbach (DE); Stefan Glorius, Konstanz (DE); Sophie Rössler, Dresden (DE)

(73) Assignee: INNOTERE GMBH, Radebeul (DE)

(21) Appl. No.: 13/394,145

(22) PCT Filed: Sep. 3, 2010
(86) PCT No.: PCT/EP2010/062951

§ 371 Date: May 9, 2012

(30) Foreign Application Priority Data
Sep. 4, 2009 (DE) 10 2009 041 248.4

(51) Int. Cl. A61F 2/28; B05D 7/00

(52) U.S. Cl. 623/23.53; 623/23.6; 427/2.24

(57) ABSTRACT

The invention relates to methods for producing a partial or complete bioactive coating of an iron and/or zinc based metal implant material with calcium phosphates, a bioactively coated iron and/or zinc based metal implant, which is partially or completely coated with calcium phosphates, and bone implants containing an implant material according to the invention. In order to produce the coating according to the invention, iron and/or zinc based metal implant materials are brought in contact with acidic aqueous solutions, which have a pH value of 6.0 or less and contain calcium phosphates, whereby a calcium phosphate layer is deposited on the surface of the implant materials. The iron and/or zinc based metal implant materials, which are used in methods according to the invention, are materials consisting of base iron alloys or pure iron or materials that contain other substances, which are coated with pure iron, with a base iron alloy and/or with zinc.

The invention concerns methods for producing a partial or complete bioactive coating of calcium phosphates on an iron-based and/or zinc-based metallic implant material and bioactively coated iron-based and/or zinc-based metallic implant materials that are partially or completely coated with calcium phosphates.

The corrosion of a metallic implant material after implantation can be desirable because in this case no removal of the implant is required after complete healing. The corrosion of metallic materials is not constant. Usually, corrosion at the beginning is strongest and decreases slowly over time because, as a result of the corrosion process (anodic metal dissolution), a passivation layer of, inter alia, sparingly soluble metal hydroxides and metal oxides is formed on the surface of the metal.

The compounds that are released upon corrosion (primarily metal ions, hydrogen, and hydroxide ions) are existing, especially immediately after implantation, in relatively high concentrations that may be toxic for the surrounding bone tissue and, in this way, may prevent ingrowth of bone tissue.

Accordingly, a medical use of corrodible metallic implants is critical because the implant, on the one hand, corrodes too quickly at the beginning and therefore has a bad tissue compatibility and, on the other hand, cannot perform a support function when it corrodes too quickly. Corrosion that is too rapid is in particular critical in case of implants of pure iron or zinc. It is therefore important to modify corrodible metallic materials in such a way that the corrosion rate is adjusted. In this connection, it is particularly important to reduce the corrosive action at the beginning, i.e., directly after implantation. Only in this way, the use of these materials as implant material is possible. In addition, the implants should be designed such that ingrowth of bone tissue is promoted in order to prevent encapsulation of the implant by connective tissue and thus implant loosening.

In order to promote integration into the bone and permanent anchoring of the implant, metallic implant materials for the bone are frequently bioactively coated. Bioactivity in this context is to be understood as the property of material to promote or trigger in (simulated) body fluid the formation of a calcium phosphate layer on a surface and, in this way, stimulate direct bonding to the bone, i.e., integration into the bone.

Clinically established are implants with so-called plasma-spray coatings in which calcium phosphate powders are heated to high temperatures in a plasma flame and applied onto the metal surface to be coated.

Newer coating processes utilize the calcium phosphate deposition from aqueous solutions wherein optionally the calcium phosphate deposition is performed by means of electrochemically enhanced processes (see, for example, U.S. Pat. No. 6,764,769, Kotte, Hofinger, Hebold). Employed metallic implant materials in this connection are titanium or titanium alloys, CoCrMo alloys or stainless steels.

Metallic implant materials disclosed in the prior art may have a solid metal structure or complex metal structures. Complex structures are, for example, porous structures, such as cellular structures.

For complex shaped metallic implants, in particular those that have a cellular structure, the coating methods that are known up to now are however insufficient. Plasma spray coatings cannot be used in principle because, as “line of sight” methods, they cannot coat undercuts.

With the known coating processes for calcium phosphates from aqueous solutions, no satisfactory results are achieved either, in particular when the coating is to be comprised of hydroxyl apatite or calcium-deficient hydroxyl apatite.

In these cases, the coating processes take a very long time and only very thin and inhomogeneous layers can be produced; U.S. Pat. No. 6,764,769 claims already layer thicknesses of >1 to 5 μm as thick coatings despite electrochemical enhancement. The layers have no homogenous surface structure because in particular calcium phosphates with high water contents are incorporated into the layers; upon drying, this leads to formation of fine inhomogeneities of the surface such as e.g. cracks.

For implants of complex metal structures and in particular cellular metal structures, there is thus no suitable method available up to now with which a homogeneous bioactive coating of calcium phosphates can be generated, in particular none with which homogenous coatings of a thickness of more than 5 pm can be generated. The reason for this limitation is the strong pH value-dependent solubility of calcium phosphates. For the direct deposition of hydroxyl apatite from aqueous solutions a pH value of >7.0 is required. At this value, however, solubility of calcium phosphates is already very low so that appropriately large quantities of aqueous solution are required in order to deposit a certain quantity of calcium phosphate. In addition, long coating periods, complex perfusion devices, electrochemical apparatus and/or complex process controls with repeated coating and drying steps are required.

Object of the invention was the development of a method for producing a partial or complete bioactive coating of an iron-based and/or zinc-based metallic implant material with calcium phosphates, the method being suitable for cellular as well as complex metal structures and, at the same time, enabling the temporal control of corrosion rate of the implant materials.

According to the invention, the object is solved by a method for producing a partial or complete bioactive coating with calcium phosphates on an iron-based and/or zinc-based metallic implant material. The coating is performed in acidic aqueous solution. For this purpose, iron-based and/or zinc-based metallic implant materials are brought into contact with acidic aqueous solutions that have a pH value of 6.0 or less and that contain calcium phosphates, whereby on the surface of the implant materials a calcium phosphate layer is deposited. The iron-based and/or zinc-based metallic implant materials used in the methods according to the invention are materials that are comprised of base iron alloys or pure iron or materials that contain other materials which are coated with pure iron, a base iron alloy and/or with zinc.

Iron-based and/or zinc-based implant materials in the meaning of the invention are referring to implant materials that contain base iron alloys or pure iron or that contain other, preferably metallic, materials that are coated with iron, an iron alloy and/or with zinc. Preferably, the iron alloys according to the invention are no stainless steel alloys. The implant materials used in the methods according to the invention are corrodible, i.e., they react and change in aqueous environment. Accordingly, the implant materials are decomposed over time.

For implant materials that contain iron or an iron alloy, coating is carried out in an acidic solution of calcium phosphates without further pretreatment and measures (except for an intensive cleaning regarding adhering contaminants such as dust or grease). For other metallic materials considered for producing implants, a prior coating of the materials with iron, an iron alloy and/or zinc greatly promotes, or even makes possible, the deposition of calcium phosphate layers from acidic calcium phosphate solutions. In particular implant materials that contain metallic materials that are not bio-corrodible before treatment with a method according to the invention must be provided with a coating with pure iron, a base iron alloy and/or zinc because the bioactive layer of calcium phosphates cannot be applied directly by a method according to the invention.

As iron-based and/or zinc-based implant materials, either materials with solid or materials with complex metal structure are suitable. Preferably, the implant materials according to the invention have a cellular metal structure. Also suitable but less preferred are solid iron-based and/or zinc-based metallic implant materials.

Surprisingly, during the course of expansive examinations of cellular structured metallic implant materials, it was found that iron-based or zinc-based metal foams in acidic aqueous solutions of calcium phosphates become coated with homogenous coatings of calcium hydrogen phosphate having the crystal structure of brushite.

Calcium phosphates mean salts that contain as cations calcium ions and as anions orthophosphate ions, metaphosphate ions and/or pyrophosphate ions, and additionally sometimes also hydrogen or hydroxide ions. Preferably, they are calcium dihydrogen phosphate (primary or monobasic calcium phosphate, calcium diphosphate, mono calcium phosphate, mono calcium dihydrogen phosphate), calcium hydrogen phosphate (secondary or dibasic calcium phosphate, also referred to in technical terminology as dicalcium phosphate), calcium phosphate (tertiary or tribasic calcium phosphate, tricalcium phosphate), tetracalcium phosphate, calcium metaphosphate, calcium diphosphate and/or apatite.

The thickness of the calcium phosphate layers can be predetermined in a targeted fashion by adjustment of the incubation conditions, in particular the composition and concentration of the solution, duration of incubation, temperature, pressure, circulation speed etc. Also, it was surprisingly found that the generated layers of calcium hydrogen phosphate even at great layer thickness can be converted into hydroxyl apatite or calcium-deficient hydroxyl apatite.

In connection with methods known from the prior art for phosphatization of iron in aqueous phosphate solutions for corrosion protection, for adhesion promotion, for friction reduction and wear reduction as well as for electrical insulation, it is known that iron phosphates are formed on the surface of iron. The surprising observation that, by contacting with acidic aqueous calcium phosphate solutions, layers of calcium phosphates can be formed was not readily deducible from the technical application of phosphatization methods for treatment of iron or steel, in particular also because one would have expected that the primary formation of a layer of iron phosphates or zinc phosphates would suppress a further deposition of calcium phosphates. The calcium phosphate layers are particularly relevant and suitable for bioactivity of bone implants.

A reason for the surprising effect that on the implant materials calcium phosphate layers are deposited must be seen in the relatively good solubility of calcium phosphates at acidic pH values (i.e., pH values of less than 6.5). Preferably, coating is therefore performed at pH values between 2.0 and 6.5. It is especially preferred that coating is carried out at pH values between 2.5 and 4.

As a result of the good solubility of calcium phosphates, coating according to the invention is preferably carried out at a relatively minimal liquid volume. Preferably, coating is carried out by contacting the metallic implant material with the aqueous solution, in particular by immersion of the implant materials in the solution. A further reason is the reaction of the iron surface in case of iron-based metallic implant materials. By oxidation of the iron in acidic medium, hydrogen is released and on the iron surface locally a pH value gradient with increased pH value at the iron surface is generated. In this way, the solubility of the surrounding calcium phosphate is reduced and this leads to deposition of calcium hydrogen phosphate on the metal surface. As a result of the substantially higher solubility of calcium phosphate at acidic pH value, the calcium phosphate deposition is significantly more effective in the coating method according to the invention as compared to conventional methods for direct deposition of hydroxyl apatite from aqueous solutions.

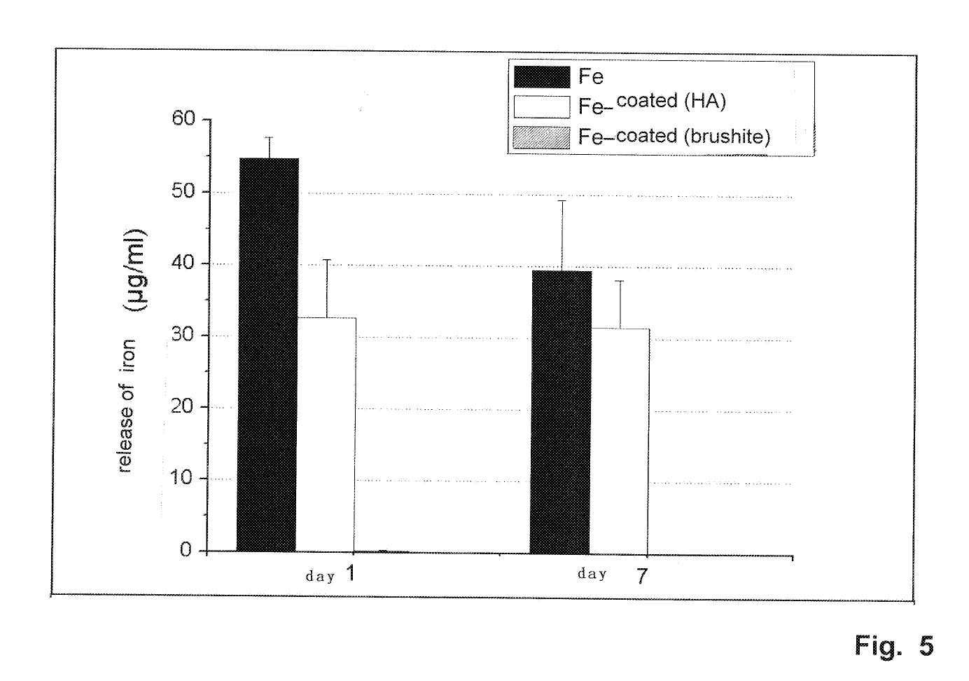

Furthermore, it was also surprisingly found that iron-based and/or zinc-based implant materials coated according to the invention from acidic calcium phosphate solution are in particular corrosion-resistant. While, for example, uncoated implant materials of ultra-pure iron in simulated body fluid and cell culture medium corrode very quickly and implant materials that are coated with hydroxyl apatite from aqueous calcium phosphate solutions exhibit only a weakly reduced corrosion rate also, for the implant materials coated according to the invention with calcium hydrogen phosphate no indication of corrosion after incubation in simulated body fluid and cell culture medium was detected (see FIG. 5). This corrosion resistance remains even when the coating with calcium hydrogen phosphate is converted secondarily into hydroxyl apatite.

These surprising results make it possible for the first time to produce implants that contain base iron alloys or pure iron or those implants that contain other, preferably metallic, materials that are coated with iron or base iron alloys and/or zinc, such implants being stable under implantation conditions even for extended period of time. In addition to the bioactivity, the bioactive coating with calcium phosphates effects thus at the same time protection against corrosion that is too fast directly after implantation. The corrosion rate of the implant material can thus be adjusted by the thickness and composition of the bioactive layer. Since the coating method according to the invention enables in an especially simple way, implant materials and implants that contain such implant materials can thus be manufactured that are producible particularly cost-efficiently.

The calcium hydrogen phosphate that is obtained as a coating is in itself already bioactive and promotes ingrowth of bone. This layer can be converted however in a simple way subsequently into hydroxyl apatite in that the implant material coated with calcium hydrogen phosphate is incubated in alkaline aqueous solution at higher pH value. For this purpose, the implant material, subsequent to coating with calcium hydrogen phosphate, is brought into contact with an alkaline solution whose pH value is at least 10 so that the deposited calcium phosphates are converted into hydroxyl apatite or calcium-deficient hydroxyl apatite.

This conversion can be done at room temperature but, in order to save time, is preferably carried out at elevated temperatures up to 100° C. By targeted selection of the conversion conditions, mixed coatings of calcium hydrogen phosphate and hydroxyl apatite can be realized also.

This is possible even for great layer thickness values of the calcium phosphates deposited beforehand (>5 μm).

The method according to the invention for producing bioactive coatings on iron-based and/or zinc-based metallic implant materials has clear advantages relative to established coating methods. For example, in contrast to plasma spray coating methods, a homogenous bioactive coating of complex and in particular cellular implant structures is even possible. No electrochemical assistance of the coating process is required. The coating can be done at room temperature but also at other environmental conditions, but in any case at conditions that are not detrimental to the implant material. The coating is realized in a short period of time and without appreciable apparatus expenditure. The achievable thickness of the coating is significantly greater than in case of electrochemically assisted coating processes. By a subsequent secondary conversion of the initially deposited layers of calcium hydrogen phosphate into hydroxyl apatite, much thicker layers of hydroxyl apatite can be produced in comparison to direct depositions of hydroxyl apatite from aqueous solutions.

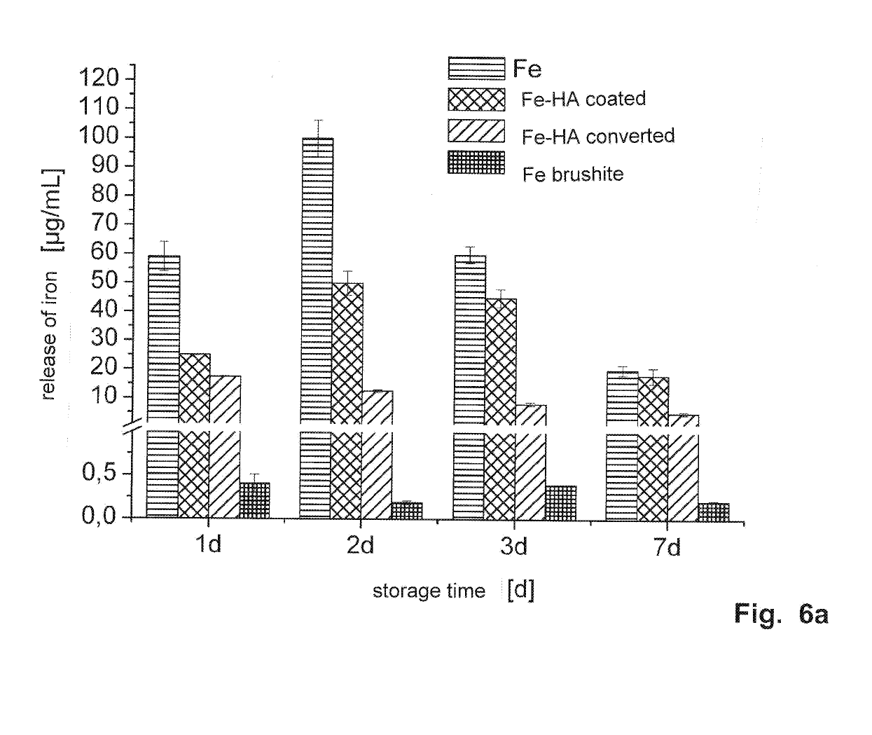

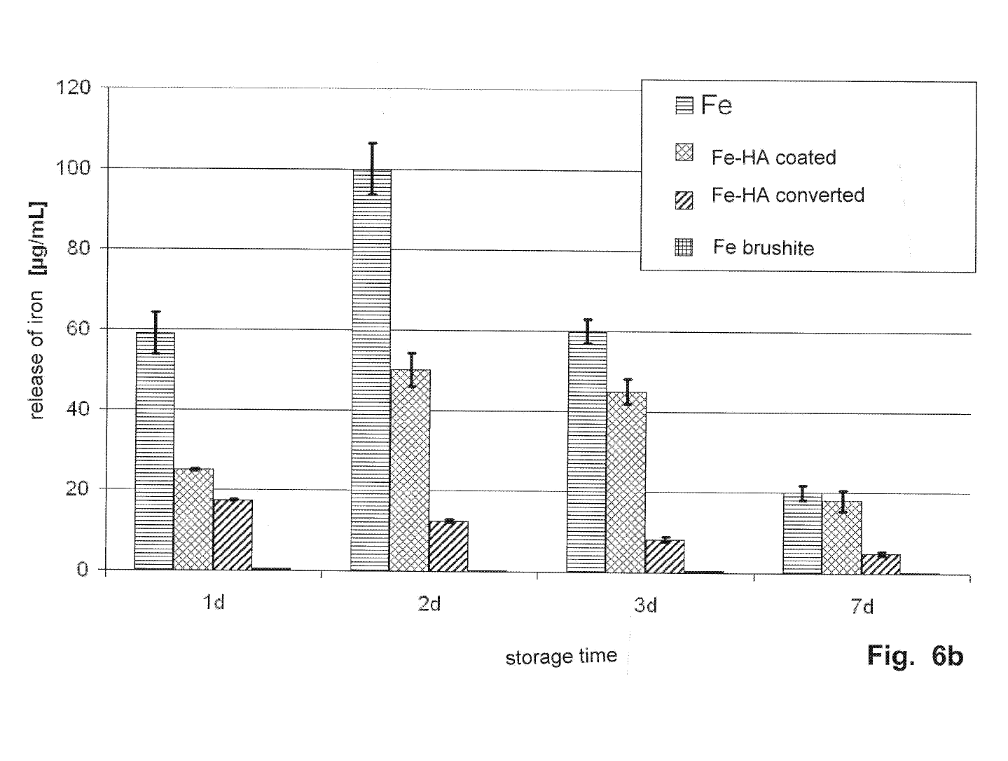

It is moreover particularly advantageous that by the coatings the corrosion behavior of the iron-based and zinc-based implant materials can be affected in a targeted way. This is not achieved in the same way by direct deposition of hydroxyl apatite on the same implant materials (compare FIG. 6).

An aspect of the invention are also the bioactively coated iron-based and/or zinc-based metallic implant materials produced by the method according to the invention.

An aspect of the invention is also a bioactively coated iron-based and/or zinc-based metallic implant material, i.e., a metallic implant material that consists of base iron alloys or pure iron or contains other materials, coated with pure iron, a base iron alloy and/or with zinc, and that is partially or completely coated with calcium phosphates. In this connection, the implant material contains in addition to the calcium phosphates a proportion of iron phosphate, in case of iron-based metallic implant materials, or a proportion of zinc phosphate, in case of zinc-based metallic implant materials.

In this connection, the layer of calcium phosphate has preferably a thickness of on average more than 5 μm. The surface of the calcium phosphate coating is homogeneous. It has a uniform layer thickness and a uniform surface structure without defects.

The implant material according to the invention is obtainable in that the surface of the metallic implant material was coated with a bioactive calcium phosphate coating in an acidic aqueous solution that has a pH value of 6.0 or less and that contains calcium phosphates. When coating according to the invention an iron-based and/or zinc-based metallic implant material in acidic aqueous solutions that contain calcium phosphates, iron phosphate or zinc phosphate is formed during the manufacturing process.

The calcium phosphate coating of the implant material according to the invention comprises preferably calcium hydrogen phosphate having the crystal structure of brushite. Already this layer of calcium hydrogen phosphate obtained by coating in acidic aqueous calcium phosphate solution is in itself bioactive so as to promote ingrowth of bone.

The layer of calcium hydrogen phosphate can be converted in a simple way by incubation in alkaline aqueous solution (at a pH value of at least 10) into hydroxyl apatite. Therefore, the calcium phosphate coating of the implant material according to the invention contains hydroxyl apatite in a preferred embodiment of the invention.

By a targeted selection of the conversion conditions also mixed coatings of calcium hydrogen phosphate and hydroxyl apatite can be realized. Therefore, the calcium phosphate coating of the implant material according to the invention contains especially preferred more than 50% hydroxyl apatite.

The coating of the implant material contains in the dried state a mass of at least 0.1 mg calcium phosphate per cm² of coated implant surface. In an advantageous embodiment of the invention, the coating of the implant material in the dried state contains a mass of at least 1.0 mg calcium phosphate per cm² of coated implant surface.

Object of the invention are also bone implants which contain at least one bioactively coated implant material in accordance with the invention. Bone implants according to the invention contain preferably different implant materials, i.e., for example, materials assembled of several parts with solids and complex metal structures of which at least one is an implant material according to the invention. Therefore, the bone implant is preferably comprised only partially of a bioactively coated implant material. In addition to the implant material according to the invention any other, preferably also metallic, shaped parts that are connected fixedly to the implant material according to the invention may be contained in the bone implant according to the invention. Appropriate shaped parts and connecting possibilities are known in the prior art.

Examples of bone implants are joint protheses that are largely comprised of solid metal structures and have structured or porous surfaces at places where their intimate and lasting connection with bone is particularly important. Artificial hip shafts have for this purpose often porous structures in the proximal area and hip sockets also porous structures in the area that is facing the bone.

Preferably, the areas of the bone implants according to the invention that are to be intimately connected to the bone are comprised of a non-corrodible metal, in particular titanium, which has a coating of pure iron, a base iron alloy, or zinc on which a bioactive coating with calcium phosphates has been deposited in accordance with the invention. The surface of the bone implant contains in this case also a proportion of iron phosphates in case of iron-based metals in the coating and a proportion of zinc phosphates in the coating in case of zinc-based metals. An advantage of the bone implants according to the invention is that an intimate connection between metal and bioactive coating is ensured.

A bone implant in the meaning of the invention is a shaped body that is partially or completely consisting of metal and is implanted at least partially in direct contact with the bone. The outer shape is essentially discretionary and depends mainly on the type of use. The shaped bodies can be a reproduction of bones or bone parts and serve for repairing bone damage or for replacement of bones or bone parts in human medicine and veterinary medicine. They can be implanted temporarily or permanently.

In one embodiment of the invention, the bone implant contains a bioactively coated implant material which has a cellular metal structure whose porosity before bioactive coating with calcium phosphates is >10%.

In one embodiment of the invention, the bone implants contain preferably also parts or segments of a bioactively coated implant material with cellular metal structures that, before coating with calcium phosphates, have a porosity of >10%.

Bone implants according to the invention contain preferably several implant materials according to the invention that are fixedly connected with each other and of which at least two have a cellular metal structure with different porosity, respectively. Preferred in this connection are bone implants that have a graded porosity i.e., the porosity at different section planes of the bone implant according to the invention is different and in particular decreases from one side to the other.

Based on attached illustrations, embodiments of the invention will be explained in more detail. In this connection, it is shown in:

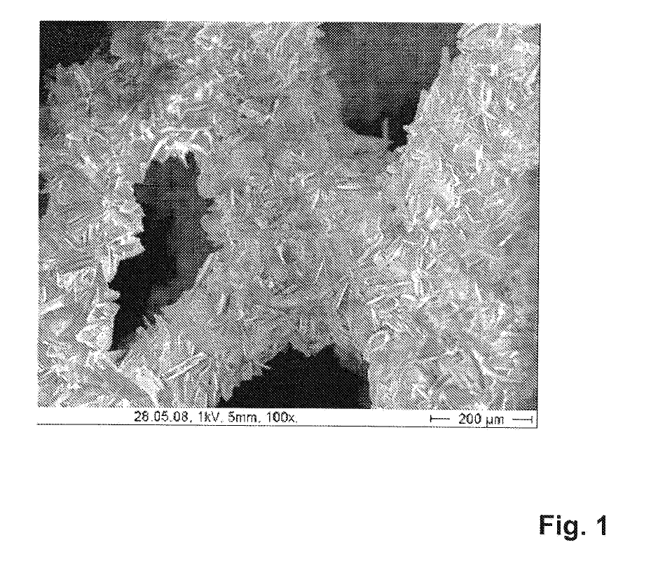

FIG. 1 SEM image (scale 200 μm) of webs of an open-pore iron foam that is coated according to the method of the invention according to example 1 with calcium hydrogen phosphate. The crystals of calcium hydrogen phosphate cover the webs of the iron foam uniformly.

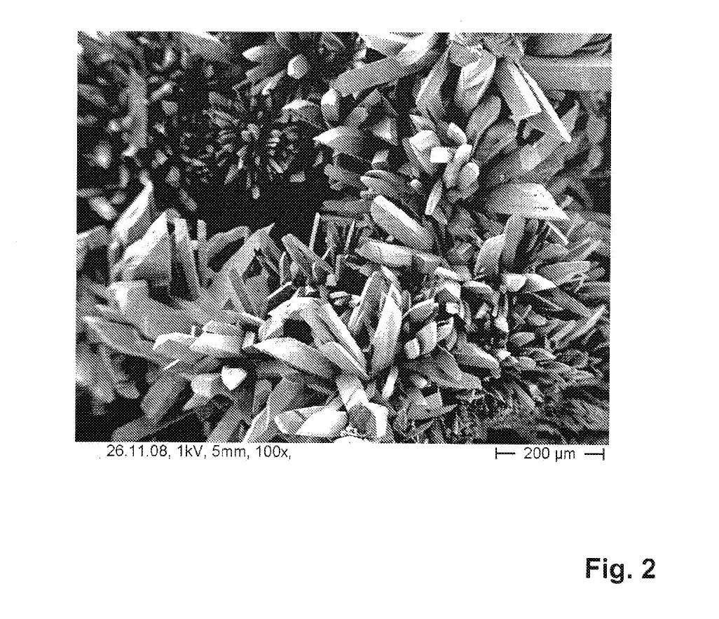

FIG. 2 SEM image (scale 200 μm) of webs of an open-pore iron foam that has been coated according to the invention according to example 1 with calcium hydrogen phosphate. The coating was carried out for a longer period of time in comparison to FIG. 1. The crystals of calcium hydrogen phosphate cover the webs of the iron foam uniformly at a thickness of approximately 100 μm.

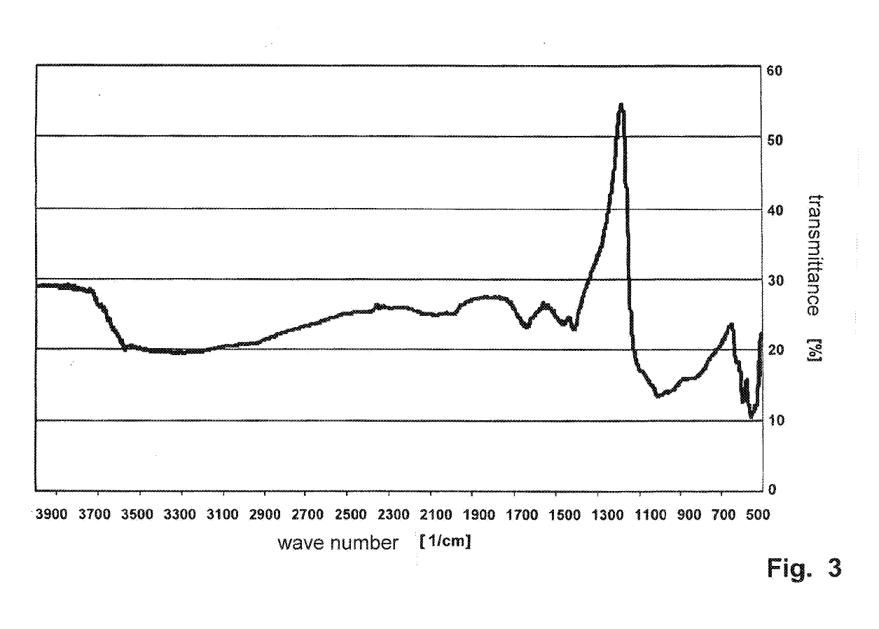

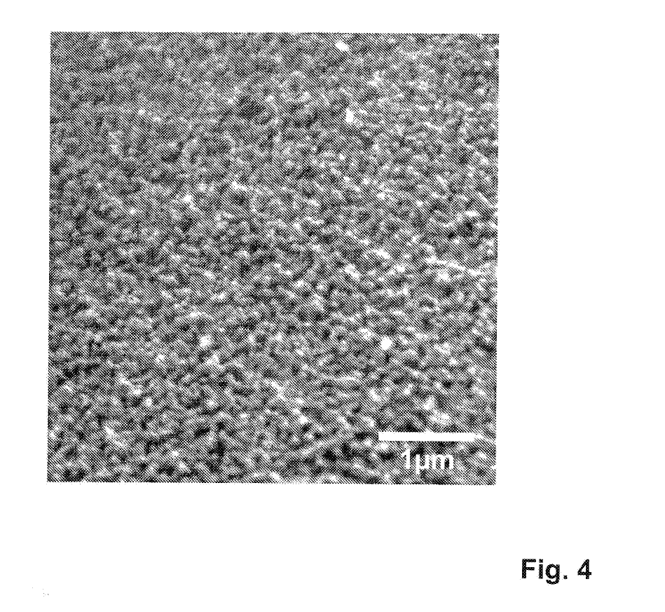

FIG. 3 FTIR analysis (Fourier transformation infrared spectroscopy) of the coating on an iron foam coated in accordance with the invention. The iron foam was first coated according to the invention with calcium hydrogen phosphate (brushite) and subsequently incubated with an alkaline aqueous solution in accordance with the invention. The analysis confirms that the homogeneous layer of calcium hydrogen phosphate is converted completely into hydroxyl apatite.

FIG. 4 SEM image (scanning electron microscope, scale 1 μm) of the surface of iron foam coated according to the invention. In analogy to FIG. 3, the iron foam was first coated according to the invention with calcium hydrogen phosphate (brushite) and subsequently incubated in alkaline aqueous solution in accordance with the invention. The image shows the fine crystal structure of hydroxyl apatite. The coating is homogeneous and conversion is complete.

FIG. 5 release of iron in cell culture medium with 15% FCS (fetal calf serum). The coating of the cellular iron foam cylinders (diameter 10 mm, height 4.5 mm, 45 pores per inch) with calcium hydrogen phosphate (Fe-coated brushite) reduces the release of iron practically completely while the hydroxyl apatite coating (Fe-coated HA) has only a minimal effect on the release of iron. Indicated is the release of iron after one day in cell culture medium (day 1) and after a week in cell culture medium (day 7).

FIG. 6 shows the release of iron in cell culture medium with 15% FCS (fetal calf serum) of differently coated cellular iron foam cylinders (diameter 10 mm, height 4.5 mm, 45 pores per inch). Illustrated are as comparative example uncoated iron foam cylinders (Fe) and iron foam cylinders with a direct hydroxyl apatite coating (Fe-HA coated), respectively. In comparison thereto, the iron foam cylinders coated according to the invention with calcium hydrogen phosphate (Fe-brushite) and hydroxyl apatite (Fe-HA converted) are illustrated. Indicated is the release of iron after 1, 2, 3 and 7 days (d) after storage in cell culture medium.

For clarification of the results, two diagrams with different size axes are illustrated (a and b).

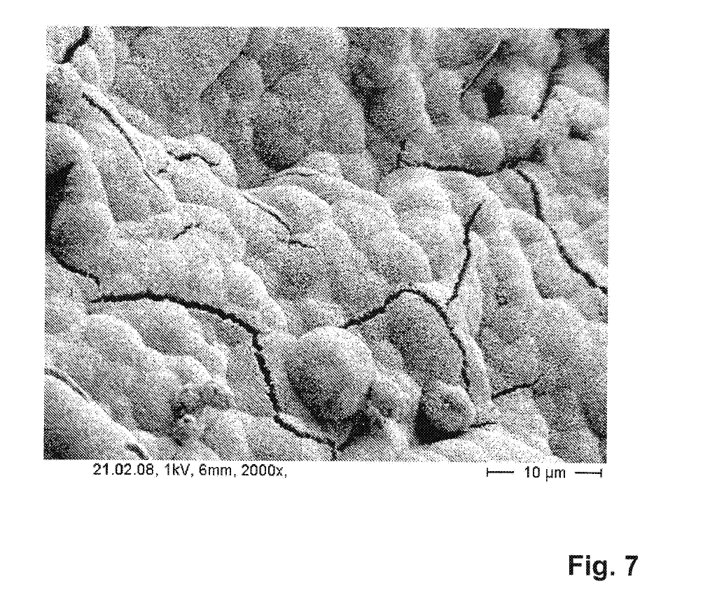

FIG. 7 comparative example: SEM image (scale 10 μm) of an iron foam that has been coated according to conventional methods with hydroxyl apatite (described in example 4). The surface is homogenous but, as can be seen in FIG. 6, this coating protects the iron foam less well in respect to corrosion in comparison to the foam cylinders coated according to the invention. The cracks in the coating are caused by the sample preparation work required for the SEM image.

### EXAMPLE 1

### Coating with Calcium Hydrogen Phosphate

A cylinder of a cellular iron foam with the dimension of 10 mm in diameter and a height of 20 mm, a purity of >99.95%, a pore width of 45 ppi (pores per inch, pores per inch) and a total porosity of 93% is incubated in 200 ml of a saturated calcium phosphate solution (Ca(H₂PO₄)) at a pH value of approximately 3.1 at room temperature for approximately 16 hours in vacuum (0.1 bar residual pressure). Subsequently, the cylinder is rinsed in DI water and dried. The weight increase is approximately 500 mg and corresponds to approximately 30% relative to the initial weight. Relative to the total surface area of the metal foam of approximately 250 cm² the load is approximately 2 mg/cm². The SEM image (FIG. 2) and a phase analysis of the calcium phosphate by means of FTIR showed that brushite (CaHPO₄×H₂O) has been deposited on the surface. Mechanical testing of the coated and uncoated metal foam cylinders resulted on average in the same compression strength values of approximately 6.2 MPa.

### EXAMPLE 2

### Conversion of Calcium Hydrogen Phosphate Coating Into Hydroxyl Apatite

Coated metal foam of example 1 is incubated in 300 ml of an 0.1 N NaOH solution for 24 hours at 95° C. Subsequently, the metal foam is rinsed with DI water and dried. The phase analysis of the converted calcium phosphate by means of FTIR shows the spectrum of hydroxyl apatite (FIG. 3). The weight reduction of the coating of approximately 200 mg corresponds to the calculated value of stoichiometric conversion of CaHPO₄×H₂O (brushite) into Ca₅(PO₄)₃OH (theoretical formula for hydroxyl apatite). The SEM image of the thus converted coating shows the fine crystal structure of hydroxyl apatite (FIG. 4). The coating is homogenous and conversion is complete.

### EXAMPLE 3

### Corrosion Behavior

Metal foam cylinders (diameter 10 mm, height 4 mm) of ultra-pure iron (99.95% Fe) with a pore width of 45 ppi were coated either in aqueous calcium phosphate solution with hydroxyl apatite or according to the method of the invention according to example 1 with calcium hydrogen phosphate. Samples of uncoated iron foam (Fe), iron foam coated with hydroxyl apatite (Fe-coated HA, see example 4 conventional method for HA coating), and iron foam coated in accordance with the invention with calcium hydrogen phosphate (Fe-coated brushite) were incubated in cell culture medium with 15% FCS at 37° C. The quantity of released iron was measured as a measure of the corrosion rate. The uncoated iron foam exhibited the highest corrosion rate, followed by the corrosion rate of the hydroxyl apatite-coated iron foam that is only a little less. The iron foam that is coated with calcium hydrogen phosphate showed almost no release of iron and can therefore be viewed as practically corrosion-resistant (FIG. 5).

### EXAMPLE 4

### Comparison of Corrosion Behavior and Quality of Coating of Iron Foam After Coating With Conventional Method and Method According to the Invention

For comparing the coating method according to the invention with a conventional method for coating of metallic implant materials with hydroxyl apatite, cellular iron foams (dimension 10 mm in diameter and height 20 mm, a purity of >99.95% Fe, pore width of 45 per inch) were coated differently.

For conventional coating, the iron foam was incubated in a first step for 3 hours in 200 ml of an alkali phosphatizing solution (preparation of the alkali phosphatizing solution: titration of 0.05% H₃PO₄ (pH 1.2) with a 1% NaH₂PO₄ dihydrate solution in a volume ratio 1:3 (H₃PO₄ to NaH₂PO₄ dihydrate solution) to a pH value of 3.5). Subsequently, the iron foam was rinsed thoroughly with deionized water, then immediately transferred into 200 ml of a 10-fold concentrated TAS solution (formulation according to Tas & Bhaduri, 2004) and incubated for further 3 hours. Subsequently, the iron foam was rinsed thoroughly with deionized water and subsequently with ethanol (p.a.) and then dried.

For coating in accordance with the invention, a cellular iron foam was coated in analogy to example 1. By doing so, layers of calcium hydrogen phosphate are produced on the iron foam. Iron foam coated in this way was treated in analogy to example 2 so that the surface coating with calcium hydrogen phosphate was converted into hydroxyl apatite.

The analysis by scanning electron microscope (SEM) shows that both coating procedures lead to coatings of hydroxyl apatite. The conventional coating method leads to characteristic crystal forms of hydroxyl apatite. The coating method according to the invention leads to layers of hydroxyl apatite that, as a result of the process, exhibit forms of brushite plates; scanning electron microscope images (FIG. 4) and by FTIR analysis (FIG. 3) however confirm that it is hydroxyl apatite.

The corrosion behavior of the differently coated iron foams was examined also in an experiment in analogy to example 3 (FIG. 6). This experiment demonstrates that the implant materials coated in accordance with the invention whose surface exhibits calcium hydrogen phosphates having the crystal structure of brushite exhibit an extremely minimal corrosion. The release of iron ions (cytotoxic in high concentrations) is extremely low already at the beginning. This is particularly advantageous for ingrowth of bone tissue into the implant material. After conversion of the calcium hydrogen phosphates into hydroxyl apatite in accordance with the method according to the invention, the implant corrodes faster again but still for all days of measurement, even at the beginning, significantly less than an uncoated or conventionally hydroxyl apatite-coated iron foam. The implant material that is coated by a conventional method with hydroxyl apatite can achieve on the first day of measurement a corrosion that is comparable to that of the implant material coated with hydroxyl apatite in accordance with the invention but already after 2 to 3 days it is apparent that the implant material coated with hydroxyl apatite in accordance with the invention is significantly more corrosion-resistant.

After 7 days it was observed that the conventional hydroxyl apatite coating effects no corrosion protection anymore. The conventionally coated implant material corrodes at the same level as the untreated iron foam. At this point in time, approximately 20 μg/ml iron are released from the conventional and the uncoated implant material. In in vitro examinations it was determined that such concentrations are cytotoxic to human mesenchymal stem cells (precursor cells of bone cells).

The corrosion of the implant material coated in accordance with invention is in contrast thereto significantly lower and is within a range that is tissue-compatible.

It has thus been demonstrated that metallic implant materials coated in accordance with the invention with calcium phosphates exhibit significantly improved corrosion properties.

### CITED NON-PATENT LITERATURE

Cuneyt Tas A, Bhaduri S B, Rapid coating of Ti₆Al₄V at room temperature with calcium phosphate solution similar to 10×simulated body fluid, J Mater Res 19 (9) 2004, pp 2742-2749.

What is claimed is:

1. A method for producing a bone implant that has a partial or complete bioactive coating comprising calcium phosphates; the method comprising:
providing an iron-based and/or zinc-based metallic implant material that consists of base iron alloys or pure iron or contains other materials that are coated with pure iron, a base iron alloy, and/or zinc;
providing an acidic aqueous solution having a pH value of 6.0 or less and containing calcium phosphates;
contacting the implant material with the acidic aqueous solution and depositing on the surface of the implant material a calcium phosphate layer of deposited calcium phosphates.

2. The method according to claim 1, comprising at least one subsequent treatment step of contacting the implant material with an alkaline solution having a pH value of at least 10 and converting the deposited calcium phosphates of the calcium phosphate layer into hydroxyl apatite or calcium-deficient hydroxyl apatite.

3. A bone implant comprising a bioactively coated iron-based and/or zinc-based metallic implant material, wherein the iron-based and/or zinc-based metallic implant material consists of base iron alloys or pure iron or contains other materials, that are coated with pure iron, a base iron alloy and/or with zinc, and wherein the implant material is coated partially or completely with a coating of calcium phosphates, and wherein the implant material exhibits a proportion of iron phosphate in case of iron-based metallic implant materials or a proportion of zinc phosphate in case of zinc-based metallic implant materials.

4. The bone implant according to claim 3, wherein the coating has a thickness of on average more than 5 μm and the surface of the coating is homogenous.

5. The bone implant according to claim 3, wherein the coating comprises calcium hydrogen phosphate having the crystal structure of brushite.

6. The bone implant according to claim 3, wherein the coating contains hydroxyl apatite.

7. The bone implant according to claim 3, wherein the coating contains more than 50% hydroxyl apatite.

8. The bone implant according to claim 3, wherein the coating in the dried state has a mass of at least 0.1 mg calcium phosphate per cm² of coated implant surface.

9. The bone implant according to claim 8, wherein the coating in the dried state has a mass of at least 1.0 mg calcium phosphate per cm² of coated implant surface.

10. The bone implant according to claim 3, wherein the implant material has a cellular metal structure whose porosity before bioactive coating with calcium phosphates is at least 10%.

11. (canceled)

12. The bone implant according to claim 3, wherein the bioactively coated implant material has a cellular metal structure whose porosity before bioactive coating with calcium phosphates is at least 10%.

13. The bone implant according to claim 3, only partially comprised of the bioactively coated implant material.

14. The bone implant according to claim 3, wherein the coating has a thickness of on average more than 5 μm.

15. The bone implant according to claim 3, wherein the surface of the coating is homogenous.
